(19) United States
(12) Patent Application Publication
(10) Pub. No.: US 2026/0272864 A1
(43) Pub. Date: Sep. 17, 2026

(54) APPLICATION OF CALCIUM DOBESILATE IN PREPARATION OF MEDICAMENT FOR PREVENTING OR TREATING CHEMOTHERAPY-INDUCED PHLEBITIS

(71) Applicant: China Pharmaceutical University, Nanjing (CN)

(72) Inventors: Jianguo Sun, Nanjing (CN); Guangji Wang, Nanjing (CN); Jiao Yan, Nanjing (CN); Ying Peng, Nanjing (CN); Yapeng Wu, Nanjing (CN); Qingqing Wang, Nanjing (CN); Wei Zhou, Nanjing (CN)

(21) Appl. No.: 19/567,545

(22) Filed: Mar. 16, 2026

(30) Foreign Application Priority Data
Mar. 17, 2025 (CN) 2025103088084

(51) Int. Cl. A61K 31/185; A61K 9/00; A61K 9/08; A61K 9/19; A61K 47/02; A61K 47/26; A61K 47/36; A61P 29/00

(52) U.S. Cl. CPC A61K 31/185; A61K 9/0019; A61K 9/08; A61K 9/19; A61K 47/02; A61K 47/26; A61K 47/36; A61P 29/00

(57) ABSTRACT

An application of calcium dobesilate in preparation of a medicament for preventing or treating chemotherapy-induced phlebitis is provided. Fluorouracil is used to induce a mouse model of the chemotherapy-induced phlebitis. It is found that intravenous injection of the calcium dobesilate not only has an advantage of rapid onset of action but also possesses vasoprotective effects. Moreover, the intravenous injection of the calcium dobesilate can significantly improve pathological manifestations of the chemotherapy-induced phlebitis and modulate expression of inflammatory cytokines, thereby demonstrating favorable protective effect against the chemotherapy-induced phlebitis.

### CROSS-REFERENCE TO RELATED APPLICATION

This application claims priority to Chinese patent application No. 202510308808.4, filed to China National Intellectual Property Administration (CNIPA) on Mar. 17, 2025, which is herein incorporated by reference in its entirety.

### TECHNICAL FIELD

The disclosure relates to an application of calcium dobesilate in preparation of a medicament for preventing or treating chemotherapy-induced phlebitis.

### BACKGROUND

The chemical name of calcium dobesilate (trade names include Doxium, Dobesil, Andomin, etc.) is calcium 2,5-dihydroxybenzenesulfonate monohydrate. As an established vasoprotective agent, the calcium dobesilate has effects of protecting vascular endothelial cells and improving microcirculation. Currently available dosage forms of the calcium dobesilate include capsules and dispersible tablets. Clinically, the calcium dobesilate is mainly used for treatment of diabetic retinopathy and has achieved good therapeutic efficacy. In recent years, applications and researches of the calcium dobesilate have gradually expanded to renal fibrosis diseases (He Shi-yang, Li Jun, “Research advances of calcium dobesilate in the treatment of renal fibrosis”, Journal of Clinical Nephrology, 2023, pp. 774-778, Vol. 23, No. 9.), atherosclerosis (Florence Njau and Hermann Haller, “Calcium Dobesilate Modulates PKCδ-NADPH Oxidase-MAPK-NF-κB Signaling Pathway to Reduce CD14, TLR4, and MMP9 Expression during Monocyte-to-Macrophage Differentiation: Potential Therapeutic Implications for Atherosclerosis”, Antioxidants, 2021, Vol. 10, No. 11, Article 1798.), etc.

Chemotherapy-induced phlebitis refers to tissue necrosis and inflammation of varying degrees caused by infusion of highly irritating chemotherapeutic drugs. Commonly used chemotherapeutic drugs in clinical practice that are prone to causing the chemotherapy-induced phlebitis include fluorouracil, vinorelbine, mitomycin, etc. As a number of cancer patients increases year by year, preventing the chemotherapy-induced phlebitis has become an urgent issue that needs to be explored and addressed. Currently, commonly used preventive measures include intravenous dexamethasone infusion, topical drug application, microwave therapy, etc. However, these measures are accompanied by problems such as corticosteroid-related side effects, cumbersome operations, and high costs.

The calcium dobesilate is clinically available only in capsules and dispersible tablets. Therefore, developing an injectable formulation that has a simple composition, low side effects, rapid onset of action, and can be pre-injected into an indwelling peripherally inserted central catheter (PICC) is of great significance for preventing the chemotherapy-induced phlebitis.

### SUMMARY

In the disclosure, fluorouracil is used to induce a mouse model of chemotherapy-induced phlebitis to investigate prophylactic protective effects of intravenous injection of calcium dobesilate on the chemotherapy-induced phlebitis. It is found that the intravenous injection of the calcium dobesilate not only has an advantage of rapid onset of action but also possesses vasoprotective effects. Moreover, the intravenous injection of the calcium dobesilate can significantly improve pathological manifestations of the chemotherapy-induced phlebitis and modulate expression of inflammatory cytokines, thereby demonstrating favorable protective effects against the chemotherapy-induced phlebitis.

An objective of the disclosure is to provide an application of the calcium dobesilate in preparation of a medicament for preventing or treating the chemotherapy-induced phlebitis.

In an embodiment, the application is an application of the calcium dobesilate in preparation of a medicament for preventing or treating the chemotherapy-induced phlebitis by reducing inflammatory cell infiltration.

In an embodiment, the application is an application of the calcium dobesilate in preparation of a medicament for preventing or treating the chemotherapy-induced phlebitis by reducing expression of interleukin-1 beta (IL-1β), interleukin-6 (IL-6), tumor necrosis factor-alpha (TNF-α), and vascular endothelial growth factor (VEGF).

In an embodiment, a dosage form of the medicament is an injection, lyophilized powder, a capsule, or a dispersible tablet.

The injection is prepared with the calcium dobesilate as an active ingredient, together with an antioxidant and water for injection, or together with the antioxidant, an osmotic pressure regulator, and the water for injection. Each 1000 milliliters (mL) of the injection contains: 1-10 grams (g) of the calcium dobesilate, 1-10 g of the antioxidant, 0-50 g of the osmotic pressure regulator, and the balance is the water for injection. Specifically, the antioxidant is selected from sodium metabisulfite or sodium sulfite. The osmotic pressure regulator is selected from sorbitol, sodium chloride (NaCl), or glucose.

In an embodiment, each 1000 mL of the injection contains: 1-10 g of the calcium dobesilate, 1-10 g of the antioxidant, and the balance is the water for injection. Alternatively, each 1000 mL of the injection contains: 1-10 g of the calcium dobesilate, 1-10 g of the antioxidant, 9-50 g of the osmotic pressure regulator, and the balance is the water for injection.

The calcium dobesilate injection of the disclosure features a simple composition, reliable efficacy, and low side effects, and can effectively prevent the chemotherapy-induced phlebitis.

In an embodiment, the lyophilized powder is an injectable formulation prepared by using the calcium dobesilate as an active ingredient and adding an appropriate amount of lyophilization protectant. Specifically, the lyophilization protectant is mannitol or dextran.

In an embodiment, a mass ratio of the calcium dobesilate to the lyophilization protectant is 1:5.

Another objective of the disclosure is to provide a method for preparing the lyophilized powder, including the following steps: dissolving the lyophilization protectant in the water for injection, followed by adding the calcium dobesilate, and stirring until completely dissolved to obtain a dissolved solution; supplementing the water for injection in the dissolved solution to adjust a final concentration of calcium dobesilate to 0.01 milligrams per milliliter (mg/mL), followed by filtering through a 0.22 micrometers (μm) filter membrane to obtain a filtered solution; filling the filtered solution into a vial, followed by performing lyophilization (i.e., freeze-drying) to obtain the lyophilized powder. Specifically, a lyophilization process is as follows.
a) A temperature of heat transfer oil in a lyophilizer (also referred to as freeze-dryer) is adjusted to −5 Celsius degrees (° C.), and the vial is placed into the lyophilizer.
b) The temperature of the heat transfer oil in the lyophilizer is adjusted to −40° C. After a sample temperature stabilizes, the temperature of the heat transfer oil is maintained for 2 hours (h)
c) Vacuum is initiated, and the temperature is raised to −15° C. (at a heating rate of 0.5 Celsius degrees per minute abbreviated as ° C./min). After a sublimation front (also referred to as waterline) of the lyophilized product disappears, the temperature of the heat transfer oil is raised to 30° C. within 2 h.
d) When the product temperature reaches 25° C., a middle isolation valve is closed for 2 minutes (min). When the vacuum in a front chamber increases by no more than 2 pascals (Pa), the product can be removed from the chamber.

In an embodiment, the application includes prophylactic administration of the calcium dobesilate prior to administration of drugs prone to causing the chemotherapy-induced phlebitis.

In an embodiment, the application includes the prophylactic administration of the calcium dobesilate 30 min prior to the administration of the drugs prone to causing the chemotherapy-induced phlebitis.

In an embodiment, the drugs prone to causing the chemotherapy-induced phlebitis include fluorouracil, cytarabine, vinorelbine, mitomycin, doxorubicin, epirubicin, daunorubicin, mechlorethamine, cyclophosphamide, vincristine, vinblastine, cisplatin, carboplatin, paclitaxel, docetaxel, etc.

### BRIEF DESCRIPTION OF DRAWINGS

FIGS. 1A-1C illustrate appearance assessment of phlebitis in C57 mice according to the disclosure. Specifically, FIG. 1A illustrates phlebitis grade in the mice; FIG. 1B illustrates changes in tail swelling in the mice; and FIG. 1C illustrates changes in body weight in the mice. Control represents a blank control group; Model represents a model group; Cim represents a positive control drug group; CaD-L represents a low-dose calcium dobesilate group; CaD-M represents a medium-dose calcium dobesilate group; and CaD-H represents a high-dose calcium dobesilate group. Data are presented as mean±standard deviation (x̅±s), n=8 (i.e., 8 mice per group), compared with the blank control group: ****P<0.0001.

FIGS. 2A-2F illustrate comparison of hematoxylin and eosin (HE) staining of tail veins in the C57 mice (200 magnification) according to the disclosure. Specifically, FIG. 2A illustrates a HE staining result of the blank control group; FIG. 2B illustrates a HE staining result of the model group; FIG. 2C illustrates a HE staining result of the positive control drug group; FIG. 2D illustrates a HE staining result of the low-dose calcium dobesilate group; FIG. 2E illustrates a HE staining result of the medium-dose calcium dobesilate group; and FIG. 2F illustrates a HE staining result of the high-dose calcium dobesilate group.

FIGS. 3A-3F illustrate expressions of VEGF-positive cells in the tail veins of C57 mice (200 magnification) according to the disclosure. Specifically, FIG. 3A illustrates expression of VEGF-positive cells in the blank control group; FIG. 3B illustrates expression of VEGF-positive cells in the model group; FIG. 3C illustrates expression of VEGF-positive cells in the positive control drug group; FIG. 3D illustrates expression of VEGF-positive cells in the low-dose calcium dobesilate group; FIG. 3E illustrates expression of VEGF-positive cells in the medium-dose calcium dobesilate group; and FIG. 3F illustrates expression of VEGF-positive cells in the high-dose calcium dobesilate group.

FIGS. 4A-4C illustrate expressions of serum inflammatory cytokines in the C57 mice (low-dose, medium-dose, and high-dose intravenous injection). Specifically, FIG. 4A illustrates expression of IL-1β levels; FIG. 4B illustrates expression of IL-6 levels; and FIG. 4C illustrates expression of TNF-α levels. Control represents the blank control group; Model represents the model group; Cim represents the positive control drug group; CaD-L represents the low-dose calcium dobesilate group; CaD-M represents the medium-dose calcium dobesilate group; and CaD-H represents the high-dose calcium dobesilate group. x̅±s, n=8, compared with the blank control group: ***P<0.001, ****P<0.0001; compared with the model group: #P<0.05, ##P<0.01, ###P<0.001, ####P<0.0001, ns indicates no significant difference.

FIGS. 5A-5B illustrate effects of different administration routes (intravenous injection and oral gavage) on the expressions of the serum inflammatory cytokines in the C57 mice. Specifically, FIG. 5A illustrates expression of IL-6 levels; and FIG. 5B illustrates expression of IL-1β levels. Control represents the blank control group; Model represents the model group; CaD-i.v. represents an intravenous injection group; CaD-p.o. represents an oral gavage group. x̅±s, n=8, compared with the blank control group: **P<0.01; compared with the model group: #P<0.05, ##P<0.01, ###P<0.001, ns indicates no significant difference.

### DETAILED DESCRIPTION OF EMBODIMENTS

The technical solutions of the disclosure are further described below through specific embodiments.

All data during experiments are expressed as mean±standard deviation (x̅±s). Statistical results are analyzed for differences between groups by using T-test and parametric tests (one-way analysis of variance abbreviated as ANOVA) in GraphPad Prism 8 (i.e., a scientific graphing and statistical analysis software developed by GraphPad Software), with P<0.05 considered statistically significant.

### Embodiment 1

### I. Experimental Materials

### 1. Experimental Animals

80 specific pathogen-free (SPF) C57BL/6J (i.e., a standard inbred mouse substrain) male mice with 7-8 weeks old and body weight 21-25 g are used in experiments. Female mice excluded due to their smaller blood vessels with greater individual variability.

### 2. Experimental Drugs and Reagents

The experimental drugs and reagents include: a fluorouracil injection (Shanghai Xudonghaipu Pharmaceutical Co., Ltd., China; specification: 0.25 g per 10 mL), a cimetidine injection (Sinopharm Rongsheng Pharmaceutical Co., Ltd., China; specification: 0.2 g per 2 mL), calcium dobesilate (Aladdin, Cat. No. C189068-25g), normal saline (Anhui Shuanghe Pharmaceutical Co., Ltd., China; specification: 4.5 g NaCl per 500 mL), a universal tissue fixative (Wuhan Servicebio Technology Co., Ltd., China; specification: 500 mL), and an ethylenediaminetetraacetic acid (EDTA) decalcifying solution (Wuhan Servicebio Technology Co., Ltd., China; specification: 500 mL).

### 3. Drug Preparation

The fluorouracil injection (25 mg/mL) is diluted with the normal saline to a concentration of 20 mg/mL.

The cimetidine injection (100 mg/mL) is diluted with the normal saline to a concentration of 2 mg/mL.

A calcium dobesilate injection is prepared as follows. 8 milligrams (mg) of the calcium dobesilate are weighed and dissolved in 4 mL of the normal saline to prepare a calcium dobesilate solution with a concentration of 2 mg/mL. The calcium dobesilate solution is then sequentially diluted with the normal saline to concentrations of 1 mg/mL and 0.5 mg/mL to obtain the calcium dobesilate injection.

### 4. Experimental Equipment

The experimental equipment includes: sterile single-use insulin syringes (Becton Dickinson Medical Devices Co., Ltd., USA), an electronic digital caliper (Harbin Measuring & Cutting Tool Group Co., Ltd., China), a mouse tail vein injection immobilization device with vein visualization (Chuangbo Global Biotechnology Co., Ltd., China, model: GEGD-Q9G), a Powerwave X microplate spectrophotometer (BioTek Instruments, Inc., USA), a Milli-Q Gradient A10 ultrapure water system (Millipore Corporation, USA), and a Minispin mini high-speed centrifuge (Eppendorf AG, Germany).

### II. Experimental Methods

### 1. Animal Grouping

The animals are randomly divided into 6 groups: a blank control group (Control), a model group (Model), a positive control drug group (i.e., cimetidine group abbreviated as Cim), and a low-dose calcium dobesilate group (CaD-L), a medium-dose calcium dobesilate group (CaD-M), and a high-dose calcium dobesilate group (CaD-H), with 8 mice in each group. Additionally, 4 groups are set up: a blank control group (Control), a model group (Model), an intravenous injection group (CaD-i.v.), and an oral gavage group (CaD-p.o.), with 8 mice in each group.

### 2. Dose Selection

Dosing regimens are determined based on clinical doses of calcium dobesilate tablets, the cimetidine injection, and the fluorouracil injection, combined with doses used in relevant literature. Specifically, low, medium, and high doses of the calcium dobesilate injection for intravenous injection in the mice are determined to be 5 milligrams per kilogram (mg/kg), 10 mg/kg, and 20 mg/kg (calculated as the calcium dobesilate), respectively. The oral gavage dose is 10 mg/kg. The intravenous dose of the cimetidine injection in the mice is 40 mg/kg (calculated as the cimetidine). The intravenous dose of the fluorouracil injection in the mice is 200 mg/kg (calculated as the fluorouracil). The above three drugs are used as a test drug (i.e., calcium dobesilate), a positive control drug (i.e., cimetidine), and a modeling drug (i.e., fluorouracil), respectively, with an administration volume of 10 milliliters per kilogram (mL/kg) for all.

### 3. Selection of the Positive Control Drug

In addition to acting as an H₂-receptor antagonist to inhibit gastric acid secretion, the cimetidine also exerts anti-inflammatory and immunomodulatory effects by inhibiting expression of IL-1β and E-selectin. Therefore, the cimetidine is widely used in treatment of skin diseases and vascular disorders. Notably, Zhuo Wang et al. reported that the cimetidine alleviates vinorelbine-induced chemotherapy-induced phlebitis in mice by inhibiting the expression of the E-selectin (Zhuo Wang et al., “Cimetidine attenuates vinorelbine-induced phlebitis in mice by militating E-selectin expression”, 2014, Cancer Chemotherapy and Pharmacology, pp. 239-247, Vol. 74, No. 2.).

### 4. Modeling Method

For the positive control drug group, the low-dose calcium dobesilate group, the medium-dose calcium dobesilate group, the high-dose calcium dobesilate group, and the oral gavage group, 20 mg/mL fluorouracil injection is administered via tail vein injection at 10 mL/kg (equivalent to 200 mg/kg) once daily for three consecutive days to establish a stable mouse model of chemotherapy-induced phlebitis. The blank control group is injected with the normal saline via the tail vein at 10 mL/kg once daily for three consecutive days.

### 5. Administration Method

For the positive control drug group, 4 mg/mL cimetidine injection is administered via tail vein injection at 10 mL/kg (equivalent to 40 mg/kg). For the low-dose calcium dobesilate group, the medium-dose calcium dobesilate group, and the high-dose calcium dobesilate group, 0.5 mg/mL, 1 mg/mL, and 2 mg/mL (equivalent to 5 mg/kg, 10 mg/kg, and 20 mg/kg) calcium dobesilate injections are injected, respectively. For the oral gavage group, the oral gavage dose is 10 mg/kg. All administrations are performed once daily for three consecutive days, with modeling performed 30 minutes after the last administration.

### 6. Observation Indicators

### 6.1 Appearance Assessment

### 6.1.1 Phlebitis Grading

Immediately after injection of the chemotherapy drug, blood vessels constrict and turn white, accompanied by manifestations such as animal restlessness and struggling. The resistance during intravenous injection gradually increases with prolonged injection time. After injection, the local vein at the injection site and surrounding tissues become hardened, while the blank control group shows no significant changes. According to a standard established by the Infusion Nurses Society (INS) and adapted to characteristics of animal experiments by excluding pain-related indicators, the phlebitis is classified into 5 grades as follows.
Grade 0: no symptoms.
Grade 1: erythema at the infusion site.
Grade 2: erythema and edema at the infusion site.
Grade 3: erythema, edema, and formation of a cord-like vein (also referred to as palpable venous cord) at the infusion site.
Grade 4: erythema, edema, formation of a cord-like vein, and purulent discharge at the infusion site.

The phlebitis is considered present when the grade is Grade 1 or higher.

### 6.1.2 Swelling Measurement

The drug doses are calculated based on body weight of the mice and administered. After modeling, the electronic digital caliper is used to measure a tail diameter of the mouse to observe the swelling degree. The swelling degree after modeling is significantly higher than that of the blank control group, with a statistically significant difference. Therefore, the modeling is considered successful.

The swelling degree is calculated according to the following formula:

Swelling (%)=(tail diameter of the mouse on day n−tail diameter of the mouse before first administration)/tail diameter before first administration×100%

### 6.1.3 Body Weight Changes

Before daily administration, the body weight of the mice is measured as a basis for dosing and as an auxiliary indicator for inflammation assessment.

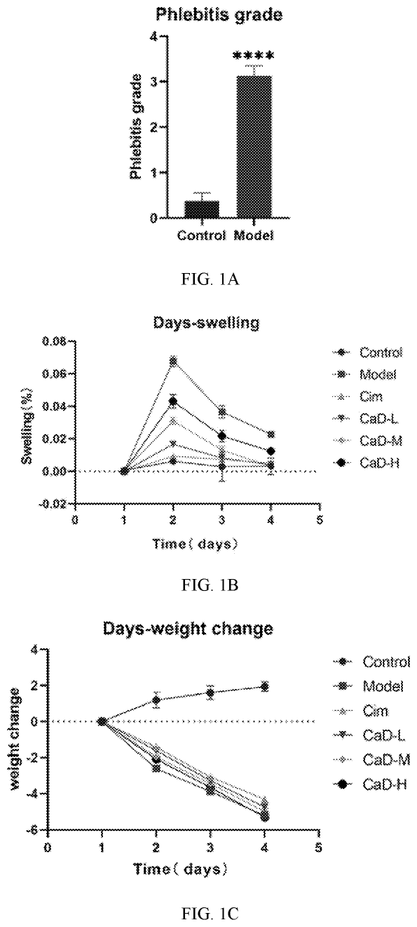

The results are shown in FIGS. 1A-1C. The phlebitis grade in a same batch reached grade 3 or above. According to the phlebitis grading standard, Grade 1 and above indicates successful model establishment. The swelling degree and body weight change results show that in the model group, tail swelling degree significantly increases and the body weight significantly decreases, whereas the calcium dobesilate prophylaxis groups show a significant trend toward recovery.

### 6.2 Microscopic Observation

To more directly evaluate effects of the calcium dobesilate on the chemotherapy-induced phlebitis in the mice, the venous edema and inflammatory cell infiltration are observed through HE staining, and VEGF is simultaneously selected for immunohistochemical analysis.

After blood collection and euthanasia, 1.5 cm of tail tissue is harvested from each mouse and immediately fixed in paraformaldehyde for 24 h. The samples are then subjected to decalcification in the EDTA decalcifying solution for 9 days. Subsequently, paraffin sections are prepared for HE staining, and immunohistochemical staining is performed to analyze expression levels of VEGF. An optical microscope is used to observe tail vein injury, the inflammatory cell infiltration, thrombosis, and tissue edema in the mice.

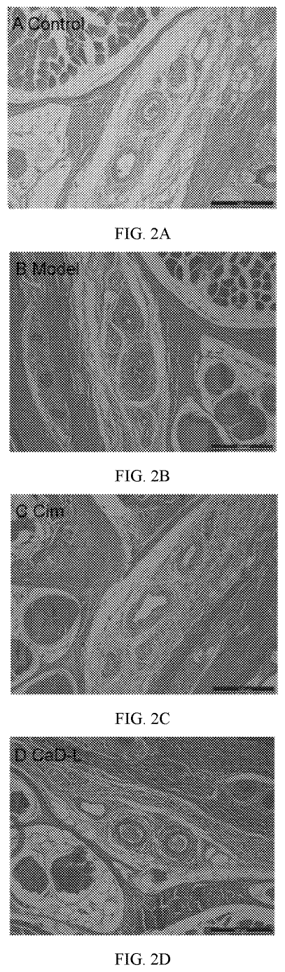

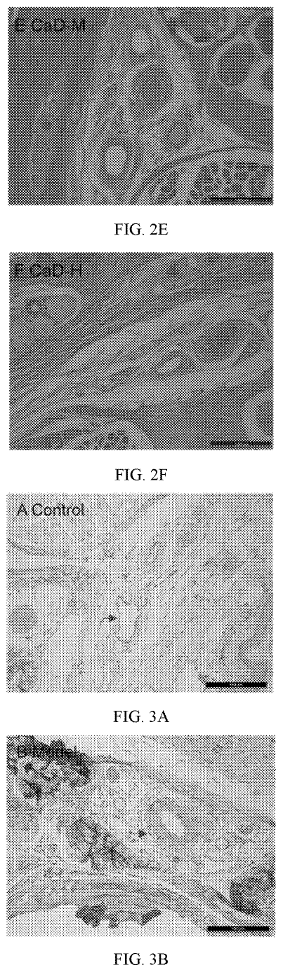

HE staining allows direct observation of a degree of venous wall edema, an extent of the inflammatory cell infiltration, and the thrombosis in the mice. Inflammatory cell nuclei, being larger, are typically stained blue, dark red granular masses in the blood vessels represent thrombi (FIG. 2A, FIG. 2B, and FIG. 2F), and light red patchy areas represent residual blood stasis (FIG. 2D, and FIG. 2E).

The HE staining results are shown in FIGS. 2A-2F. Compared with the blank control group, the mice in the model group exhibited severe venous wall edema and extensive inflammatory cell infiltration. Intervention with the calcium dobesilat and the positive control drug ameliorates these symptoms, resulting in reduced edema and mild inflammatory infiltration.

The immunohistochemical analysis can assess disease severity based on intensity of brownish-yellow staining. VEGF is a diffusible endothelial cell-specific mitogen and a vascular permeability factor that acts on endothelial cells to promote mitosis, thereby inducing angiogenesis. Meanwhile, VEGF exerts a significant effect in enhancing vascular permeability, leading to increased exudation. VEGF is generally expressed at low levels in normal adult humans and animals. Its expression level is related to a degree of endothelial cell injury repair, severity of the inflammation, and conditions such as the thrombosis (Yang Wenqin et al., “Clinical observation of potato juice combined with dexamethasone in preventing fluorouracil-induced phlebitis”, National Seminar on New Advances in Oncology Nursing, Chinese Nursing Association, 2011, pp. 339-341).

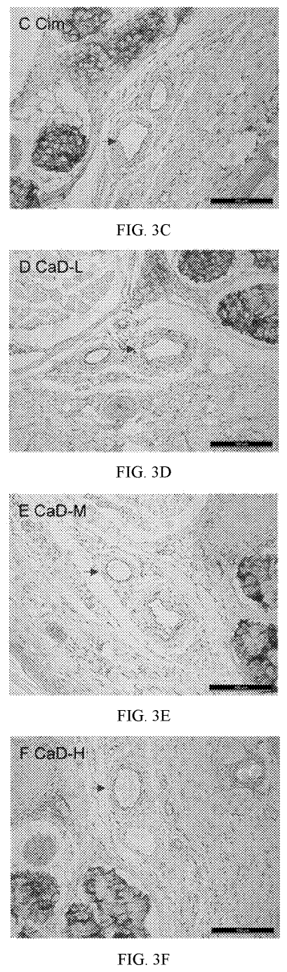

Immunohistochemical results are shown in FIGS. 3A-3F. Compared with the blank control group, expression of VEGF-positive cells in the mice is significantly increased in the model group. The intervention with calcium dobesilate and the positive control drug restores positive expression to normal levels.

### 6.3 Enzyme-Linked Immunosorbent Assay (ELISA) Analysis

Blood samples are collected from the mice via retro-orbital bleeding. A 50 microliters (μL) blood sample is placed into a dry tube, and then centrifuged to isolate serum. An upper layer of the serum is used to detect levels of the inflammatory cytokines, including IL-1β, TNF-α, IL-6, etc.

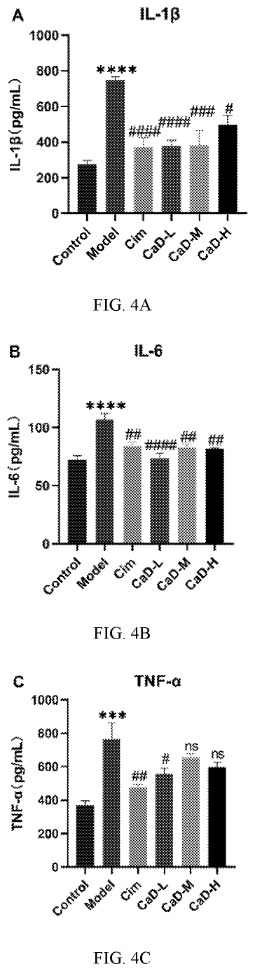

As shown in FIGS. 4A-4C, expression of serum inflammatory cytokines in the mice is detected by ELISA. Compared with the blank control group, levels of IL-1β, IL-6, and TNF-α in the mice are significantly increased in the model group. Pretreatment with the calcium dobesilate significantly reverses the expression levels of these inflammatory cytokines.

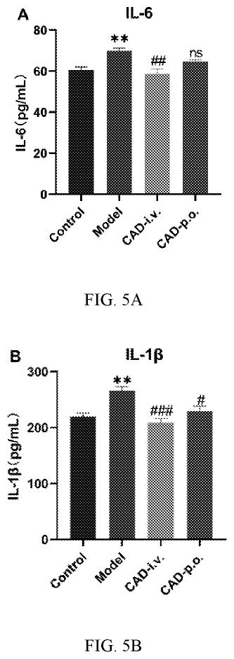

As shown in FIGS. 5A-5B, compared with the model group, the calcium dobesilate intravenous injection group (i.e., CaD-i.v.) shows significantly reduced levels of IL-1ß and IL-6 (P<0.01) in the mice. The oral gavage group shows no trend toward reversal of IL-6 levels compared with the model group, but shows a significant decreasing trend for IL-1ß (P<0.05). These results indicate that the intravenous administration has a more significant effect than the oral gavage.

In conclusion, the calcium dobesilate can ameliorate the fluorouracil-induced chemotherapy-induced phlebitis in the mice by reducing the characteristic indicators of the chemotherapy-induced phlebitis, including IL-1β, IL-6, TNF-α, and VEGF. The calcium dobesilate and the positive control drug exhibit similar anti-inflammatory effects, with the calcium dobesilate showing superior efficacy in reducing IL-6 compared to the positive control drug. This indicates that the calcium dobesilate has a significant therapeutic effect on the chemotherapy-induced phlebitis.

### Embodiment 2

A composition of a calcium dobesilate small-volume injection in this embodiment is as follows: calcium dobesilate 10 g, sodium metabisulfite 1 g, and water for injection added to 1000 mL.

A preparation method of the calcium dobesilate small-volume injection has the following steps.
Step (1), 800 mL of freshly prepared water for injection is taken and heated to 40° C., followed by adding the sodium metabisulfite, and stirring until completely dissolved to obtain a sodium metabisulfite solution.
Step (2), the calcium dobesilate is added to the sodium metabisulfite solution, followed by stirring until completely dissolved to obtain a dissolved solution.
Step (3), the water for injection is supplemented to the dissolved solution to a final volume, followed by filtering through a 0.22 μm filter membrane to obtain a filtered solution.
Step (4), the filtered solution is filled into ampoules at 5 mL per ampoule under nitrogen protection, followed by sealing, and subjecting to autoclaving at 121° C. for 15 min to obtain the calcium dobesilate small-volume injection.

### Embodiment 3

A composition of a calcium dobesilate small-volume injection in this embodiment is as follows: calcium dobesilate 10 g, sodium sulfite 2 g, sorbitol 50 g, and water for injection added to 1000 mL.

A preparation method of the calcium dobesilate small-volume injection has the following steps.
Step (1), 800 mL of freshly prepared water for injection is taken and heated to 40° C., followed by adding the sodium sulfite and the sorbitol, and stirring until completely dissolved to obtain a mixed solution.
Step (2), the calcium dobesilate is added to the mixed solution, followed by stirring until completely dissolved to obtain a dissolved solution.
Step (3), the water for injection is supplemented to the dissolved solution to a final volume, followed by filtering through a 0.22 μm filter membrane to obtain a filtered solution.
Step (4), the filtered solution is filled into ampoules at 5 mL per ampoule under nitrogen protection, followed by sealing, and subjecting to autoclaving at 121° C. for 15 min to obtain the calcium dobesilate small-volume injection.

### Embodiment 4

A composition of a calcium dobesilate large-volume injection in this embodiment is as follows: calcium dobesilate 10 g, sodium metabisulfite 10 g, NaCl 90 g, and water for injection added to 10000 mL.

A preparation method of the calcium dobesilate large-volume injection has the following steps.
Step (1), 2000 mL of freshly prepared water for injection is taken, followed by adding the sodium metabisulfite and NaCl, and stirring until completely dissolved to obtain a mixed solution.
Step (2), the calcium dobesilate is added to the mixed solution, followed by stirring until completely dissolved to obtain a dissolved solution.
Step (3), the water for injection is supplemented to the dissolved solution to a final volume, followed by filtering through a 0.22 μm filter membrane to obtain a filtered solution.
Step (4), the filtered solution is filled into infusion bottles at 50 mL per bottle under nitrogen protection, followed by sealing, and subjecting to autoclaving at 121° C. for 20 min to obtain the calcium dobesilate large-volume injection.

### Embodiment 5

A composition of a calcium dobesilate large-volume injection in this embodiment is as follows: calcium dobesilate 10 g, sodium sulfite 10 g, glucose 500 g, and water for injection added to 10000 mL.

A preparation method of the calcium dobesilate large-volume injection has the following steps.
Step (1), 2000 mL of freshly prepared water for injection is taken, followed by adding the sodium sulfite and the glucose, and stirring until completely dissolved to obtain a mixed solution.
Step (2), the calcium dobesilate is added to the mixed solution, followed by stirring until completely dissolved to obtain a dissolved solution.
Step (3), the water for injection is supplemented to the dissolved solution to a final volume, followed by filtering through a 0.22 μm filter membrane to obtain a filtered solution.
Step (4), the filtered solution is filled into infusion bottles at 50 mL per bottle under nitrogen protection, followed by sealing, and subjecting to autoclaving at 121° C. for 20 min to obtain the calcium dobesilate large-volume injection.

### Embodiment 6

A composition of calcium dobesilate lyophilized powder for injection in this embodiment is as follows: calcium dobesilate 10 g, and mannitol 50 g.

A preparation method of the calcium dobesilate lyophilized powder has the following steps.
Step (1), 800 mL of freshly prepared water for injection is taken, followed by adding the mannitol, and stirring until completely dissolved to obtain a mannitol solution.
Step (2), the calcium dobesilate is added to the mannitol solution, followed by stirring until completely dissolved to obtain a dissolved solution.
Step (3), the water for injection is supplemented to the dissolved solution to a final volume of 1000 mL to obtain a diluted solution.
Step (4), the diluted solution is filtered through a 0.22 μm filter membrane, followed by filling into vials at 5 mL per vial to obtain bottled solutions.
Step (5), the bottled solutions are performed lyophilization to obtain the calcium dobesilate lyophilized powder, with a lyophilization process as follows.
a) A temperature of heat transfer oil in a lyophilizer is adjusted to −5° C., and the vials are placed into the lyophilizer.
b) The temperature of the heat transfer oil in the lyophilizer is adjusted to −40° C. After a sample temperature stabilizes, the temperature of the heat transfer oil is maintained for 2 h.
c) Vacuum is initiated, and the temperature is raised to −15° C. (at a heating rate of 0.5° C./min). After a sublimation front of the lyophilized product disappears, the temperature of the heat transfer oil is raised to 30° C. within 2 h.
d) When the product temperature reaches 25° C., a middle isolation valve is closed for 2 min. When the vacuum in a front chamber increases by no more than 2 Pa, the product can be removed from the chamber.
e) Stoppering: after lyophilization, a vacuum pump is turned off, clean nitrogen is introduced into the front chamber, and the product is stoppered.
Step (6), the product is removed from the chamber and crimped.

### Embodiment 7

A composition of calcium dobesilate lyophilized powder for injection in this embodiment is as follows: calcium dobesilate 10 g, and dextran 50 g.

A preparation method of the calcium dobesilate lyophilized powder has the following steps.
Step (1), 800 mL of freshly prepared water for injection is taken and heated to 40° C., followed by adding the dextran, and stirring until completely dissolved to obtain a dextran solution.
Step (2), the calcium dobesilate is added to the dextran solution, followed by stirring until completely dissolved to obtain a dissolved solution.
Step (3), the water for injection is supplemented to the dissolved solution to a final volume of 1000 mL to obtain a diluted solution.
Step (4), the diluted solution is filtered through a 0.22 μm filter membrane, followed by filling into vials at 5 mL per vial to obtain bottled solutions.
Step (5), the bottled solutions are performed lyophilization to obtain the calcium dobesilate lyophilized powder, with a lyophilization process as follows.
a) A temperature of heat transfer oil in a lyophilizer is adjusted to −5° C., and the vials are placed into the lyophilizer.
b) The temperature of the heat transfer oil in the lyophilizer is adjusted to −40° C. After a sample temperature stabilizes, the temperature of the heat transfer oil is maintained for 2 h.
c) Vacuum is initiated, and the temperature is raised to −15° C. (at a heating rate of 0.5° C./min). After a sublimation front of the lyophilized product disappears, the temperature of the heat transfer oil is raised to 30° C. within 2 h.
d) When the product temperature reaches 25° C., a middle isolation valve is closed for 2 min. When the vacuum in a front chamber increases by no more than 2 Pa, the product can be removed from the chamber.
e) Stoppering: after lyophilization, a vacuum pump is turned off, clean nitrogen is introduced into the front chamber, and the product is stoppered.
Step (6), the product is removed from the chamber and crimped.

What is claimed is:

1. An application of calcium dobesilate in preparation of a medicament for preventing chemotherapy-induced phlebitis, wherein drugs causing the chemotherapy-induced phlebitis comprise fluorouracil, cytarabine, vinorelbine, mitomycin, doxorubicin, epirubicin, daunorubicin, mechlorethamine, cyclophosphamide, vincristine, vinblastine, cisplatin, carboplatin, paclitaxel, and docetaxel; and
wherein a dosage form of the medicament is an injection or lyophilized powder.

2. The application as claimed in claim 1, comprising:
using the calcium dobesilate as an active ingredient together with an antioxidant and water for injection to prepare the injection; or
using the calcium dobesilate together with the antioxidant, an osmotic pressure regulator, and the water for injection;
wherein each 1000 milliliters (mL) of the injection contains: 1-10 grams (g) of the calcium dobesilate, 1-10 g of the antioxidant, 0-50 g of the osmotic pressure regulator, and the balance is the water for injection; the antioxidant is selected from sodium metabisulfite or sodium sulfite;
and the osmotic pressure regulator is selected from sorbitol, sodium chloride (NaCl), or glucose.

3. The application as claimed in claim 2, wherein each 1000 mL of the injection contains: 1-10 g of the calcium dobesilate, 1-10 g of the antioxidant, and the balance is the water for injection; or each 1000 mL of the injection contains: 1-10 g of the calcium dobesilate, 1-10 g of the antioxidant, 9-50 g of the osmotic pressure regulator, and the balance is the water for injection.

4. The application as claimed in claim 1, wherein the lyophilized powder is an injectable formulation prepared by using the calcium dobesilate as an active ingredient and adding an appropriate amount of a lyophilization protectant; and the lyophilization protectant is mannitol or dextran.

5. The application as claimed in claim 4, wherein a mass ratio of the calcium dobesilate to the lyophilization protectant is 1:5.
